(19) United States
(12) Patent Application Publication
(10) Pub. No.: US 2026/0239558 A1
(43) Pub. Date: Aug. 13, 2026

(54) PORTABLE SMOKE DISPERSAL DEVICE FOR SMUDGING

(71) Applicant: Charleston Financial Group, LLC, Milton, GA (US)

(72) Inventor: Robert Hicks, Milton, GA (US)

(21) Appl. No.: 19/536,628

(22) Filed: Feb. 11, 2026

Related U.S. Application Data
(60) Provisional application No. 63/757,708, filed on Feb. 12, 2025.

(51) Int. Cl. H05K 7/20

(52) U.S. Cl. CPC H05K 7/20172

(57) ABSTRACT

A handheld smudging device comprises a housing configured for single-handed operation, a combustion chamber within the housing and configured to receive smudging material, and a spark capture screen positioned to cover an outlet of the combustion chamber and configured to retain burning particles while allowing smoke to pass therethrough. A fan assembly is disposed within the housing and configured to generate airflow through the combustion chamber and past the smudging material. A battery power source is configured to power the fan assembly. The combustion chamber may comprise retention features configured to secure the smudging material in a predetermined position. The spark capture screen may be removably attachable to the combustion chamber to allow access for loading and unloading the smudging material. The fan assembly may comprise a DC motor and fan blades configured to draw ambient air into the housing and direct the air across the smudging material.

### RELATED APPLICATION

Under provisions of 35 U.S.C. § 119(e), the Applicant claims benefit of U.S. Provisional Application No. 63/757,708 filed on Feb. 12, 2025, and having inventors in common, which is incorporated herein by reference in its entirety.

It is intended that each of the referenced applications may be applicable to the concepts and embodiments disclosed herein, even if such concepts and embodiments are disclosed in the referenced application with different limitations and configurations and described using different examples and terminology.

### FIELD OF DISCLOSURE

The present disclosure generally relates to portable smoke generation and distribution devices for aromatherapy, spiritual practices, and air treatment applications. More particularly, the disclosure relates to battery-powered handheld smoke dispersal apparatus incorporating controlled burning chambers, integrated safety features including spark containment mechanisms, and fan-driven airflow systems for automated and controlled smoke distribution from incense materials such as sage, palo santo, and cedar in smudging rituals and wellness environments.

### BACKGROUND

In some situations, users perform smudging rituals using incense materials such as sage, palo santo, or cedar for aromatherapy and spiritual cleansing purposes. For example, practitioners light bundles of dried sage by hand and allow the material to smolder naturally while moving through residential or commercial spaces. Thus, the conventional strategy is to manually wave the burning incense bundle or blow on the smoldering material to increase smoke output and distribute smoke throughout the desired area. This often causes problems because the conventional strategy does not provide consistent smoke production throughout the duration of the ritual. For example, the volume of smoke generated varies unpredictably based on how vigorously the user waves the bundle or blows on the embers.

The conventional strategy does not maintain a desired smoke output level. The user must continuously adjust their manual effort to compensate for changes in the smoldering rate. The conventional strategy does not provide adequate control over smoke distribution patterns.

In some situations, users perform smudging rituals using incense materials such as sage, palo santo, or cedar for aromatherapy and spiritual cleansing purposes. For example, practitioners light bundles of dried sage by hand and allow the material to smolder naturally while moving through residential or commercial spaces. Thus, the conventional strategy is to manually wave the burning incense bundle or blow on the smoldering material to increase smoke output and distribute smoke throughout the desired area. This often causes problems because the conventional strategy does not provide consistent smoke production throughout the duration of the ritual. For example, the volume of smoke generated varies unpredictably based on how vigorously the user waves the bundle or blows on the embers. The conventional strategy does not maintain a desired smoke output level. The user must continuously adjust their manual effort to compensate for changes in the smoldering rate. The conventional strategy does not provide adequate control over smoke distribution patterns.

The conventional strategy presents safety hazards during operation. Open burning of incense materials creates risks of burns when users carry the burning materials through multiple rooms. Sparks and embers can escape from the burning bundle during manual handling. The risk increases when users wave the burning material to generate additional smoke. The conventional strategy requires continuous physical effort from the user. The user must blow on the smoldering material repeatedly to maintain smoke production. The user must wave the burning bundle continuously to distribute smoke throughout a space. This physical demand becomes exhausting during extended sessions. This physical demand creates accessibility barriers for individuals with physical limitations. The conventional strategy does not provide portability for use in various locations. Traditional incense burners remain stationary during use. The user must carry open flames when moving between spaces. The conventional strategy does not allow adjustment of smoke intensity to match user preferences or space requirements.

There is a need for a solution that provides consistent smoke production without requiring continuous manual intervention from the user. There is a need for a solution that maintains uniform smoke output levels throughout extended smudging sessions. There is a need for a solution that eliminates the physical demands associated with manual smoke generation methods. There is a need for a solution that reduces safety hazards by containing sparks and embers during operation. There is a need for a solution that enables portability while maintaining safe containment of burning materials. There is a need for a solution that allows users to adjust smoke intensity according to space requirements and personal preferences. There is a need for a solution that provides controlled directional smoke dispersal without manual waving or blowing. There is a need for a solution that operates independently of electrical outlets to enable use in multiple locations. There is a need for a solution that reduces accessibility barriers for individuals with physical limitations. There is a need for a solution that integrates traditional smudging practices with modern convenience and safety standards.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

A handheld smudging device may be provided. The housing may be configured for single-handed operation. A combustion chamber may be disposed within the housing. The combustion chamber may be configured to receive smudging material. A spark capture screen may be positioned to cover an outlet of the combustion chamber. The spark capture screen may be configured to retain burning particles while allowing smoke to pass therethrough. A fan assembly may be disposed within the housing. The fan assembly may be configured to generate airflow through the combustion chamber and past the smudging material. A battery power source may be configured to power the fan assembly.

The combustion chamber may comprise retention features. The retention features may be configured to secure the smudging material in a predetermined position within the combustion chamber. The spark capture screen may be removably attachable to the combustion chamber. The removable attachment may allow access for loading and unloading the smudging material. The fan assembly may comprise a DC motor and fan blades. The DC motor and fan blades may be configured to draw ambient air into the housing. The DC motor and fan blades may be configured to direct the air across the smudging material. The housing may comprise thermal insulation. The thermal insulation may be positioned between the combustion chamber and a grip area. The thermal insulation may prevent heat transfer to a user's hand.

A user interface may be disposed on the housing. The user interface may be configured to control operation of the fan assembly. The user interface may comprise a multi-speed selector. The multi-speed selector may be configured to adjust airflow velocity generated by the fan assembly. The combustion chamber may be configured to maintain smoldering of the smudging material. The smoldering may be maintained without re-igniting open flame during fan operation. The spark capture screen may comprise a mesh. The mesh may have apertures sized to prevent passage of burning fragments. The mesh may maintain acceptable pressure drop for smoke flow. The battery power source may comprise a rechargeable battery. The housing may include a charging interface.

The user interface may comprise specific control elements positioned for single-handed operation accessibility. The user interface may include a power button positioned approximately 25 mm to 50 mm from the grip center to enable thumb activation without repositioning the hand. The multi-speed selector may comprise a rotary dial or sliding switch providing 3 to 5 discrete speed settings corresponding to fan speeds of approximately 20%, 40%, 60%, 80%, and 100% of maximum RPM. The interface may include tactile feedback such as detents or clicks to provide confirmation of speed selection without requiring visual confirmation. The controls may be recessed approximately 1 mm to 3 mm below the housing surface to prevent accidental activation during handling. The interface may include LED indicators positioned adjacent to the controls to display operational status and battery charge level using different colors or blinking patterns.

A smudging system may be provided. A handheld smudging apparatus may be provided. The handheld smudging apparatus may comprise a housing. The housing may be configured for portable operation. A tubular combustion container may be configured to receive smudging material. A removable spark containment cover may be configured to attach to the tubular combustion container. A powered fan may be configured to generate directional airflow through the tubular combustion container. A portable power source may be provided. A charging system may be configured to recharge the portable power source.

The tubular combustion container may comprise heat-resistant material. The tubular combustion container may include air inlet and outlet paths for controlled airflow. The removable spark containment cover may comprise a mesh screen. The mesh screen may be configured to trap sparks and embers while allowing smoke passage. Interchangeable combustion containers may be configured for different smudging materials. The powered fan may be configured to increase smoke output per unit time compared to manual smudging techniques.

A method of smudging a space may be provided. Smudging material may be inserted into a combustion chamber of a handheld smudging device. The smudging material may be ignited to establish combustion. Open flame may be extinguished from the smudging material while maintaining smoldering. A spark capture screen may be attached over the combustion chamber. A fan may be activated within the handheld smudging device. The fan may generate airflow across the smoldering smudging material. Smoke output may be directed from the handheld smudging device toward target areas within the space.

The smoke output may be directed by orienting the handheld smudging device. The handheld smudging device may be oriented to project smoke toward corners, ceiling perimeters, and doorways. Fan speed may be adjusted to control smoke volume and projection distance. The smudging material may comprise at least one of sage, palo santo, cedar, and sweetgrass. Smoke output may be monitored. The smudging material may be re-ignited when smoke production diminishes. The spark capture screen may be maintained in position during re-ignition.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 depicts a perspective view of a portable smoke dispersal device.

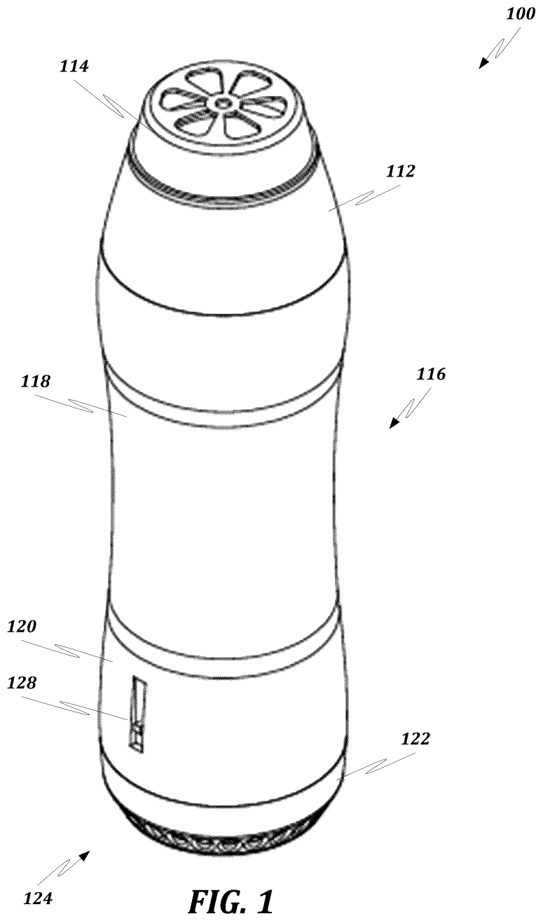

FIG. 2 depicts a front elevation view of the portable smoke dispersal device.

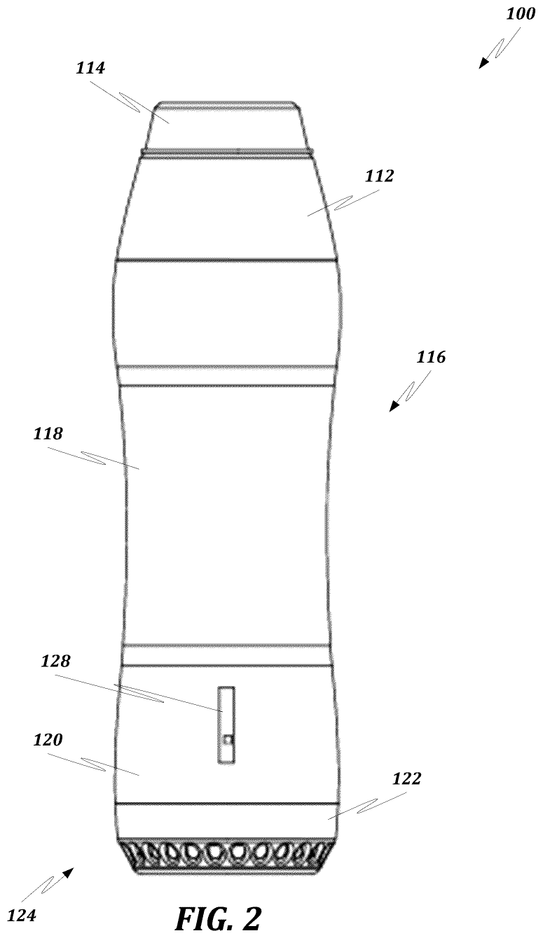

FIG. 3 depicts a side elevation view of the portable smoke dispersal device.

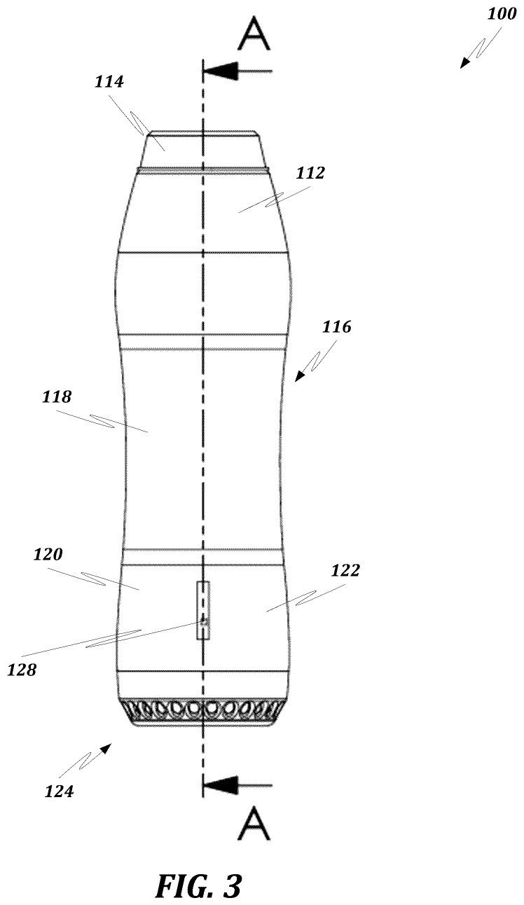

FIG. 4 depicts a longitudinal cross-sectional view of the portable smoke dispersal device taken along line A-A of FIG. 3.

FIG. 5 depicts an exploded perspective view of the portable smoke dispersal device showing component separation.

FIG. 6 depicts a flowchart illustrating a method of dispersing smoke using the portable smoke dispersal device.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely to provide a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such a term to mean based on the contextual use of the term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subject matter disclosed under the header.

The present disclosure addresses technical challenges encountered in smoke generation and dispersal for smudging ceremonies and/or aromatherapy applications. Traditional manual methods of burning incense materials may present limitations in consistency, control, safety, and efficiency that may impact the effectiveness of smudging practices across diverse environments and user needs.

Manual smudging methods may rely on natural smoldering of incense materials such as sage, cedar, or palo santo. The smoke production rate in such methods may be subject to variations in ambient air currents, material moisture content, and environmental conditions. A user performing traditional smudging may need to repeatedly blow on the smoldering material to maintain adequate smoke output. This manual intervention may result in uneven smoke distribution throughout a space. The smoke volume generated by natural smoldering alone may be insufficient for larger rooms or multi-room environments. High ceilings and complex architectural layouts may further complicate smoke dispersal using conventional techniques.

The portable smoke dispersal device 100 in the present disclosure may address this problem through integration of a battery-operated fan with the incense container tube. The fan may generate controlled airflow through the container tube, passing over the smoldering incense material. This forced airflow may maintain consistent smoke production without requiring manual intervention. The fan-driven dispersal may produce a directed smoke stream that may be more effectively distributed throughout a space compared to naturally rising smoke. The handheld configuration of the device 100 may allow a user to direct the smoke output to specific locations, including corners, high ceiling areas, and spaces behind furniture that may be difficult to reach with traditional methods.

Traditional smudging practices may involve carrying openly burning or smoldering incense materials through indoor spaces. Sparks and embers may be expelled from the burning material during movement. These expelled particles may pose fire hazards when they contact flammable surfaces such as carpets, curtains, or furniture. The risk may be elevated when smudging is performed in unfamiliar spaces or by inexperienced practitioners. Hot ash falling from the incense material may cause burns to the user or damage to surfaces below.

The device 100 may incorporate multiple containment features to address these safety concerns. The mesh screen positioned within the container tube may capture sparks and embers while allowing smoke to pass through. The openings in the mesh screen may be sized in a range of approximately 0.5 mm to 2 mm, which may be small enough to trap ember-sized particles while permitting smoke flow. The removable spark cover may provide an additional barrier at the open end of the container tube. This spark cover may include perforations that allow smoke egress while preventing larger particles from escaping. The dual-layer containment system comprising the internal mesh screen and external spark cover may substantially reduce the risk of ember escape during device operation.

The container tube may be constructed from heat-resistant materials such as metal or ceramic. These materials may withstand the elevated temperatures generated by smoldering incense without degradation or deformation. The housing may provide thermal insulation between the hot incense container and the user's hand on the grip area. This thermal isolation may prevent burns during extended use periods.

Manual smudging methods may provide minimal control over smoke output intensity. A user may attempt to adjust smoke production by blowing harder or more frequently on the smoldering material. This approach may be physically tiring during extended smudging sessions. The smoke intensity achieved through manual methods may vary significantly based on the user's lung capacity and technique. Different spaces and applications may require different smoke densities, but traditional methods may not easily accommodate such variations.

The device 100 may address this limitation through incorporation of variable fan speed control. The control switch mounted on the handle portion may be configured as a multi-position switch allowing selection between discrete fan speeds. In one embodiment, the switch may provide low, medium, and high speed settings. The low speed setting may generate gentle airflow suitable for small rooms or subtle smoke dispersal. The medium speed setting may provide moderate airflow appropriate for average-sized residential spaces. The high speed setting may produce maximum airflow for large commercial spaces or rapid smoke generation.

The controller operatively coupled to the fan may regulate the voltage or current supplied to the fan motor to achieve the selected speed. A microcontroller implementation may provide precise speed control and may enable additional features such as programmable speed profiles. The user may adjust the fan speed during operation without interrupting the smudging process. This real-time adjustability may allow the user to respond to changing needs as they move through different areas of a space.

Traditional smudging techniques may require specific knowledge and physical capabilities. Proper technique for lighting and maintaining smoldering incense may need to be learned through practice. The physical act of repeatedly blowing on incense may be difficult for individuals with respiratory limitations. Elderly users or those with mobility restrictions may find it challenging to carry traditional smudging implements while moving through spaces. The learning curve associated with traditional methods may discourage some individuals from engaging in smudging practices.

The device 100 may lower the barrier to entry for smudging practices through its simplified operation. The method of using the device 100 may provide a straightforward procedure that may be easily learned. This simplified procedure may eliminate the need for specialized breathing techniques or repeated manual intervention. The device 100 may perform the smoke generation and dispersal functions automatically once activated. Users with limited physical capabilities may operate the device 100 with minimal exertion. The consistent results provided by the device 100 may reduce the skill level required to achieve effective smudging outcomes. The present disclosure includes many aspects and features. Moreover, while many aspects and features relate to, and are described in, the context of a handheld electric incense burning device, embodiments of the present disclosure are not limited to use only in this context.

### I. Platform Overview

This overview is provided to introduce a selection of concepts in a simplified form that are further described below. This overview is not intended to identify key features or essential features of the claimed subject matter. Nor is this overview intended to be used to limit the claimed subject matter's scope.

The portable smoke dispersal device may provide a modernized approach to the ancient practice of smudging, combining traditional materials with contemporary technology to enhance the ritual while maintaining its cultural significance. This handheld device may comprise a container tube for holding incense material such as sage or palo santo, integrated with a battery-powered fan. The container tube may include a mesh screen configured to capture any sparks or embers from the burning incense while still allowing smoke to pass through. A removable spark cover may also be included to further contain any embers after the incense is lit.

The handheld nature of the device may allow the user to easily maneuver it throughout a space, directing smoke into corners, up towards high ceilings, or around furniture. This may enable more thorough and efficient coverage compared to traditional manual methods. The consistent airflow from the fan may also help maintain even smoke production for the duration of the smudging ritual.

The ability to control fan speed may allow users to adjust the rate of smoke production and dispersal as needed for different room sizes or desired smudging intensities. The perforated screen within the incense material container may capture sparks and embers while still allowing smoke to pass through. This may reduce the risk of accidental burns or fires compared to handling openly burning incense materials.

The self-contained, battery-powered design may allow for easy transport and use in multiple locations without being tethered to a power outlet. The battery-powered design may enable continuous smoke production and dispersal for longer periods compared to manual methods that rely on human breath. This may be particularly advantageous for smudging larger spaces.

By containing the burning incense material within the device, it may reduce the spread of ash or debris compared to openly carrying smoldering materials. The consistent smoke output may enable more standardized and repeatable smudging practices, which may be beneficial in professional settings like spas or wellness centers.

Embodiments of the present disclosure may comprise components including, but not limited to, at least one of the following:
A. A Housing
B. A Container Tube
C. A Spark Cover
D. A Fan With Controller
E. A Power Source

Details with regards to each component are provided below. Although components are disclosed with specific functionality, it should be understood that functionality may be shared between components, with functions split between components, and/or duplicated by the components. Furthermore, the name of each component should not be construed as limiting upon the functionality of the component.

The following depicts an example of a method of a plurality of methods that may be performed by at least one of the aforementioned components. Although methods and/or steps may be described to be performed by a single component, it should be understood that, in some embodiments, different operations may be performed by different components in operative communication with one another.

Furthermore, although the stages of the following example method are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in orders that differ from the ones disclosed below. Moreover, various stages may be added or removed without altering or departing from the fundamental scope of the depicted methods and systems disclosed herein.

Consistent with embodiments of the present disclosure, a method may be performed by at least one of the components disclosed herein. The method may comprise the following stages:
inserting smudging material into a combustion chamber of a handheld smudging device;
igniting the smudging material to establish combustion;
extinguishing open flame from the smudging material while maintaining smoldering;
attaching a spark capture screen over the combustion chamber;
activating a fan within the handheld smudging device to generate airflow across the smoldering smudging material; and
directing smoke output from the handheld smudging device toward target areas within the space.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

### II. Platform Configuration

The portable smoke dispersal device disclosed herein may represent a technological advancement in smudging and incense burning practices by integrating battery-powered fan technology with traditional ceremonial smoke generation. The device may address fundamental limitations of conventional manual smudging methods, including inconsistent smoke production, safety hazards from ember escape, limited reach and coverage, and physical demands on users during extended ceremonies. By combining a handheld housing with an elongated incense receptacle, dual ember containment system, variable-speed fan assembly, and intelligent control features, the device may provide controlled, directed smoke dispersal while maintaining the authentic spiritual and cultural aspects of traditional smudging practices.

The device may include a dual ember containment architecture, which may comprise an internal mesh spark screen positioned within the incense receptacle and a removable external spark cap covering the open end. This redundant safety system may capture embers and sparks at multiple stages while allowing smoke to pass through freely, substantially reducing fire hazards associated with carrying smoldering incense materials through indoor spaces. The internal spark screen may feature precisely sized openings between approximately 0.5 mm and 2 mm to trap ember-sized particles while maintaining optimal smoke flow, while the external spark cap may include perforations that permit smoke egress while preventing larger particle escape. This dual-layer approach may provide enhanced safety compared to traditional open-flame smudging methods.

The device's fan-assisted smoke generation system may create consistent, directed airflow that maintains smoldering without requiring manual intervention such as repeated blowing on the incense material. The battery-powered fan may draw ambient air through the incense receptacle, entraining smoke produced by naturally smoldering stick-form incense materials and projecting it as a controlled stream. Variable speed control may allow users to adjust smoke output intensity for different applications, from gentle dispersal in small spaces to vigorous output for large commercial environments. The fan-driven approach may enable users to direct smoke to previously inaccessible areas such as high ceilings, corners, and spaces behind furniture, expanding the effective coverage area compared to naturally rising smoke.

The device's ergonomic handheld design may facilitate single-handed operation during ceremonial movement, with the handle portion configured for comfortable gripping during extended use. The twist-lock mechanism coupling the incense receptacle to the housing may enable easy disassembly for cleaning and maintenance while providing secure attachment during operation. Sealing elements may prevent air leakage to maintain consistent airflow performance. The rechargeable battery system may provide cordless portability, allowing unrestricted movement through spaces without concern for power cord limitations. Automatic safety features such as thermal protection and programmable shut-off timers may prevent overheating and conserve battery power while ensuring safe operation.

The device may optionally include an integrated electrical heating coil as an ignition system powered by the battery power source. The heating coil may comprise a resistive heating element such as nichrome wire configured to generate heat when electrical current is applied. The coil may be controlled by a separate switch or button on the user interface, allowing the user to activate heating independently of fan operation. The heating coil may draw approximately 10 to 50 watts of power and may reach ignition temperature within approximately 10 to 30 seconds of activation. The coil may include automatic shut-off after a predetermined heating period of approximately 30 seconds to 2 minutes to prevent overheating and conserve battery power. When the heating coil is integrated into the device, the battery capacity may be increased to accommodate the additional power requirements for both fan operation and ignition heating.

When a separate electrical coil device is used for ignition, the coil device may be configured to interface with the incense container or may be inserted into the combustion chamber to contact the smudging material directly. The separate coil device may comprise a handheld heating tool with its own power source, such as a rechargeable battery or plug-in electrical connection. The separate coil may include a probe or heating element sized to fit within the combustion chamber opening without requiring removal of the spark capture screen. For powder material ignition, the separate coil device may include a flat heating plate or coil configuration that can be pressed against the powder surface to initiate smoldering. The separate coil approach may provide ignition capability without adding complexity or power requirements to the main smudging device.

This technological approach may preserve the essential characteristics of traditional smudging while addressing practical limitations that may restrict its use in modern environments. The device may burn authentic incense materials in their natural smoldering state without active heating, maintaining the cultural and spiritual significance of the practice. The enhanced safety features, consistent performance, and improved accessibility may make smudging practices available to a broader range of users, including those with physical limitations or limited experience with traditional techniques. Professional applications in wellness centers, spas, and therapeutic environments may benefit from the device's ability to provide consistent, controlled smoke coverage while maintaining operational efficiency and safety standards.

Accordingly, embodiments of the present disclosure provide a platform comprised of a distributed set of components, including, but not limited to:

### A. A Housing

The smudging device 100 may include a housing 116. The housing 116 may provide structural support and containment for the internal components. The housing 116 may be configured as a generally cylindrical body. The cylindrical body may extend along a longitudinal axis. The housing 116 may comprise a fixed sidewall component 118. The fixed sidewall component 118 may define the outer perimeter of the device 100. The fixed sidewall component 118 may be constructed from a durable material, such as (but not limited to) plastics, metals, and/or composite materials. The fixed sidewall component 118 may provide rigidity to the overall structure, helping to maintain the alignment of internal components during operation.

The housing 116 may further comprise a rotatable adjustment component 112. The rotatable adjustment component 112 may be disposed adjacent to the fixed sidewall component 118. The rotatable adjustment component 112 may be configured to rotate relative to the fixed sidewall component 118. The rotation may occur about the longitudinal axis of the housing 116. The rotatable adjustment component 112 may be configured to engage an engagement section of the container tube 102. The rotatable adjustment component 112 may allow a user to adjust the axial position of the container tube 102 relative to the housing 116. The adjustment may be achieved by rotating the rotatable adjustment component 112. The axial translation may position the incense material at a desired location relative to the spark cover 110.

In some embodiments, the rotatable adjustment component 112 may include threading or other features extending radially inward from an interior surface. The threading or other features may engage with complementary threading on the exterior of the container tube 102. The threaded engagement may convert rotational motion into linear motion. The linear motion may move the container tube 102 upward or downward within the housing 116. The rotatable adjustment component 112 may include grip features on an exterior surface. The grip features may facilitate manual rotation by a user. The grip features may comprise ridges, knurling, or textured surfaces.

The housing 116 may include a base 122. The base 122 may be located at a lower end of the housing 116 to create a stable platform for the device 100. The base 122 may define airflow apertures 124 distributed around a circumference of the base. The airflow apertures 124 may allow ambient air to enter the housing 116. The ambient air may be drawn into the housing 116 by the fan 126. The airflow apertures 124 may be sized to provide adequate airflow. The sizing may balance airflow volume with structural integrity of the base 122. The airflow apertures 124 may be shaped as circular openings, slots, or perforations. The shape may be selected to optimize air intake while preventing ingress of debris.

The base 122 may include a lower surface. The lower surface may contact a support surface when the device 100 is placed in an upright position. The lower surface may be flat or slightly contoured. The contour may provide stability on uneven surfaces. The base 122 may include feet or standoffs to elevate the lower surface above the support surface. The elevation may improve airflow into the airflow apertures 124. The feet or standoffs may be constructed from a non-slip material. The non-slip material may prevent the device 100 from sliding during operation.

The housing 116 may include internal mounting structures (e.g., brackets, ribs, bosses, etc.) to secure the fan 126 within the housing 116. The mounting structures may position the fan 126 to direct airflow through the container tube 102. The housing 116 may define a battery compartment housing the power source 130. In some embodiments (e.g., where the power source 130 is a removable battery), the battery compartment may be accessible via a removable cover secured with fasteners and/or a snap-fit mechanism. The battery compartment may include electrical contacts that connect the power source 130 to the fan 126.

In some embodiment (e.g., where the power source 130 is a rechargeable battery), the housing 116 may include a charging port (not shown), such as a USB interface or other charging interface. The charging port may be located on the fixed sidewall component 118 or the base 122. The charging port may be covered by a protective flap when not in use.

The housing 116 may include a control interface region. The control interface region may be located on the fixed sidewall component 118. The control interface region may provide access to the control switch 128. The control switch 128 may be mounted flush with or protrude from the exterior surface of the housing 116. The control interface region may include labeling or indicators identifying the function and/or state of the control switch 128.

The fixed sidewall component 118 may include a handle region contoured to fit a user's hand. The contour may provide ergonomic grip during operation. The handle region may include a non-slip coating or texture to enhance grip security. The handle region may be located at a position that balances the weight of the device 100 to help reduce user fatigue during extended use.

The housing 116 may be configured for single-handed operation through specific dimensional and ergonomic parameters. The overall length of the housing 116 may be in a range of approximately 200 mm to 350 mm to provide adequate grip length while maintaining portability. The grip diameter may be in a range of approximately 25 mm to 40 mm to accommodate various hand sizes. The weight distribution may be balanced such that the center of gravity is positioned within approximately 50 mm to 100 mm from the grip center to minimize wrist strain during extended use. The housing 116 may include finger indentations or contoured surfaces positioned at approximately 30 mm intervals along the grip region to provide secure finger placement. The grip surface may include a coefficient of friction of at least 0.6 when measured against dry skin to prevent slippage during operation.

In some embodiments, the housing 116 may be constructed in multiple sections. The multiple sections may be joined by fasteners, adhesives, or welding, and/or threaded engagement. The joints between sections may be sealed to prevent air leakage. The sealed joints may maintain airflow efficiency through the intended pathways. The housing 116 may include ventilation openings (separate from the airflow apertures 124) to allow heat dissipation from internal components. The heat dissipation may prevent overheating during extended operation.

### B. A Container Tube

The smudging device 100 may include a container tube 102 configured to receive and securely hold incense material during operation. The container tube 102 may comprise an elongated tubular body extending from a first end to a second end along a longitudinal axis of the device 100. The tubular body may define an internal chamber dimensioned to accommodate stick-form incense materials. The internal chamber may have a generally circular cross-section, having a diameter selected to provide a secure fit for commonly available incense materials for smudging (e.g., sticks, smudge sticks, bundled herbs, loose, herbs, etc.) while allowing adequate airflow around the smoldering material.

In some embodiments, the container tube 102 may be removably attached to the device 100 to allow for cleaning, maintenance, and replacement. The container tube 102 may include a connection mechanism such as threading, bayonet coupling, or friction fit that allows the tube to be detached from the housing 116 without tools. The removable connection may enable the user to separate the container tube 102 from the device 100 for thorough cleaning of residue and ash that may accumulate during use. The removable design may also allow for replacement of the container tube 102 if damaged or worn, or for substitution with different tube configurations optimized for specific smudging materials.

The container tube 102 may comprise an incense container 104 defining an opening through which incense material may be inserted into the internal chamber. The opening may be sized to permit insertion incense materials having various diameters. The incense container 104 may include an internal support structure configured to retain the incense material in a predetermined position within the internal chamber. The internal support structure may comprise friction-fit surfaces and/or mechanical retention features (e.g., clips, grooves, ridges, etc.) formed on interior walls of the incense container 104.

The incense container 104 and container tube 102 may be configured to accommodate powder form incense in addition to stick and bundled forms. For powder applications, the container 104 may include a mesh or perforated base positioned at the bottom of the combustion chamber to support the powder while allowing airflow to pass through. The mesh base may have apertures sized to prevent powder from falling through while maintaining adequate air circulation. The container may include measurement markings or fill lines to guide proper powder loading quantities and prevent overfilling that could restrict airflow.

In some embodiments, the removable nature of the container tube 102 may provide access to internal components for maintenance and cleaning. When the container tube 102 is removed from the device 100, the user may access the fan assembly, airflow passages, and electrical connections for inspection and cleaning. The removable design may include sealing elements such as O-rings or gaskets that maintain airtight connections when assembled while allowing easy separation when removal is desired. The container tube 102 may include visual indicators or markings to guide proper installation depth and orientation when reattaching to the device 100.

The interchangeable combustion containers may be configured with standardized connection interfaces to accommodate different smudging materials. Each container may include a threaded or bayonet connection compatible with the housing engagement section, allowing tool-free replacement in approximately 5 to 15 seconds. The containers may be available in different internal diameters ranging from approximately 6 mm for thin incense sticks to approximately 30 mm for large sage bundles. The container length may vary from approximately 50 mm for short-duration applications to approximately 200 mm for extended ceremonies. Each container may include material-specific retention features optimized for the intended smudging material, such as fine grooves for powdered materials or coarse ridges for bundled materials. The containers may be color-coded or labeled to indicate the intended material type and may include capacity markings to guide proper loading quantities.

The retention features may include specific mechanical elements configured to secure various smudging material geometries. The retention features may comprise spring-loaded clips positioned at approximately 120-degree intervals around the chamber circumference, with each clip providing a retention force of approximately 1 N to 5 N. The clips may be fabricated from heat-resistant spring steel and may extend approximately 2 mm to 8 mm into the chamber interior to contact the smudging material. For cylindrical materials, the retention features may include V-shaped grooves machined into the chamber wall at approximately 10 mm intervals along the chamber length. The predetermined position may be established by positioning the material such that the burning end extends approximately 5 mm to 15 mm beyond the chamber outlet to optimize airflow interaction while maintaining secure retention during device operation and handling.

The tubular combustion container may have specific geometric characteristics optimized for different smudging materials. The tubular configuration may provide a length-to-diameter ratio in a range of approximately 3:1 to 10:1 to accommodate elongated smudging materials such as sage bundles and palo santo sticks. The internal surface may include longitudinal grooves or channels spaced at approximately 60-degree to 120-degree intervals around the circumference to provide multiple contact points for securing irregularly shaped materials. For sage applications, the container diameter may be approximately 15 mm to 25 mm to accommodate bundled sage sticks, while for palo santo applications, the diameter may be approximately 8 mm to 15 mm to accommodate individual wood sticks. The container material may be stainless steel or ceramic with a thermal conductivity low enough to prevent heat transfer to the housing while maintaining structural integrity at temperatures up to approximately 400° C.

The internal chamber may have specific dimensional parameters to accommodate various smudging materials while ensuring proper airflow. The internal diameter may be in a range of approximately 8 mm to 25 mm to accommodate stick-form incense ranging from thin sage sticks to bundled smudge sticks. The chamber length may be in a range of approximately 75 mm to 150 mm to hold standard incense lengths while providing adequate combustion space. The chamber may include internal ridges or grooves spaced at approximately 5 mm to 10 mm intervals to provide friction-fit retention of the smudging material. The chamber walls may have a minimum thickness of approximately 1 mm to 3 mm to provide structural integrity while maintaining heat resistance. The chamber may include air inlet ports having a total cross-sectional area of approximately 10% to 30% of the chamber cross-sectional area to ensure adequate oxygen supply for sustained smoldering.

The container tube 102 may comprise an outer wall 106 defining the exterior surface of the tubular body. The outer wall 106 may be constructed from a heat-resistant material. The heat-resistant material may comprise stainless steel, aluminum, brass, or high-temperature ceramic. The outer wall 106 may have a wall thickness sufficient to provide structural integrity while maintaining a compact form factor. The wall thickness may be in a range of approximately 0.5 mm to 2 mm. The outer wall 106 may include heat dissipation features. The heat dissipation features may comprise fins, grooves, or textured surfaces configured to increase surface area for convective heat transfer to ambient air.

The container tube 102 may comprise a threaded engagement section 108. The engagement section 108 may optionally allow for adjusting the location of the incense container 104 along the longitudinal axis of the device 100 by rotating the rotatable portion 112 of the housing 116. The rotatable portion 112 may include internal threading that engages with corresponding external threading on the engagement section 108. When a user chooses to rotate the rotatable portion 112, the threaded engagement may cause at least a portion of the container tube 102 to translate axially within the housing 116. This optional axial translation may move the incense container 104 closer to or farther from the spark cover 110, allowing the user to optimize the position of the smudging material relative to the airflow path and spark containment features. The adjustment mechanism may provide optional fine-tuning of the incense position to accommodate different material lengths and to optimize smoke production efficiency.

The threaded engagement section 108 may also facilitate complete removal of the container tube 102 from the device 100. By rotating the rotatable portion 112 beyond the normal adjustment range, the container tube 102 may be completely disengaged from the housing 116. This complete removal capability may allow for thorough cleaning of both the container tube 102 and the interior of the housing 116. The removable design may include alignment features or keying to ensure proper reinstallation and prevent incorrect assembly that could affect device operation or safety.

The container tube 102 may comprise a spark screen 110 positioned within the internal chamber. The spark screen 110 may be configured to capture sparks and ash particles generated by smoldering incense material while allowing smoke to pass through. The spark screen 110 may be positioned transversely across the internal chamber at an open end of the container tube 102. The spark screen 110 may divide the internal cavity into a combustion zone proximal to the incense container 104 and a smoke passage zone proximal to the threaded engagement section 108.

The spark screen 110 may comprise a perforated barrier, such as (but not limited to) a woven wire mesh or a perforated metal plate. The woven wire mesh may be constructed from stainless steel wire. The woven wire mesh may have a mesh size in a range of approximately 40 mesh to 60 mesh. The mesh size may correspond to openings having dimensions in a range of approximately 0.5 mm. The perforated metal plate may comprise circular perforations. The perforated metal plate may comprise elongated slot perforations. The perforations may have dimensions in a range of approximately 0.5 mm to 2 mm.

The spark screen 110 may be secured within the internal chamber by a mounting structure, such as an annular retaining ring. The annular retaining ring may be press-fit into the internal chamber. The annular retaining ring may engage with an annular groove formed in the inner surface of the outer wall 106. The spark screen 110 may be captured between the annular retaining ring and a shoulder formed in the internal chamber. The mounting structure may allow the spark screen 110 to be replaced when clogged with accumulated ash or when damaged.

The spark screen 110 may serve multiple functions during operation of the smudging device 100. The spark screen 110 may physically intercept burning embers dislodged from the smoldering incense material by airflow generated by the fan 126. The spark screen 110 may capture ash particles having dimensions larger than the openings in the perforated barrier. The spark screen 110 may allow smoke particles to pass through the openings due to the smaller size of smoke particles relative to the openings. The spark screen 110 may function as a heat shield by absorbing thermal energy from hot gases passing through the perforated barrier. The absorbed thermal energy may be dissipated to the outer wall 106 through conductive heat transfer. The spark screen 110 may distribute airflow more uniformly across the smoldering incense material by creating a pressure drop across the perforated barrier.

The mesh apertures may be sized according to specific dimensional criteria to balance ember retention with smoke flow efficiency. The aperture size may be in a range of approximately 0.5 mm to 2.0 mm to prevent passage of typical burning fragments while allowing smoke particles to pass freely. The mesh may be fabricated from stainless steel wire having a diameter of approximately 0.1 mm to 0.3 mm to provide adequate strength while minimizing flow restriction. The open area ratio of the mesh may be approximately 40% to 70% to maintain acceptable pressure drop of less than approximately 50 Pa at typical operating flow rates. The mesh may include a support frame to prevent deformation under thermal stress, with the frame providing structural support while maintaining the specified aperture dimensions during heating and cooling cycles.

The removable spark containment cover may include specific attachment and removal mechanisms for user accessibility. The cover may include a bayonet-style connection with tabs positioned at approximately 120-degree intervals that engage with corresponding slots in the container rim. The removal force may be approximately 3 N to 10 N applied in a combined rotational and axial motion to disengage the bayonet connection. The cover may include a heat-resistant handle or grip feature extending approximately 10 mm to 20 mm beyond the container diameter to enable safe removal when the container is warm. The containment cover may include a double-wall construction with an air gap of approximately 2 mm to 5 mm to provide thermal insulation and prevent burns during removal. The cover may include visual indicators such as alignment marks or color coding to guide proper installation and ensure complete spark containment.

The spark screen 110 may be positioned at a specific distance from the smudging material to optimize ember capture while maintaining smoke flow. The spark screen 110 may be positioned approximately 10 mm to 30 mm from the burning end of the smudging material to allow adequate smoke generation space while capturing dislodged embers. The spark screen 110 may be retained by an annular mounting ring having an interference fit of approximately 0.1 mm to 0.5 mm with the inner wall of the container tube 102. The mounting ring may include tabs or detents that engage with corresponding grooves in the container tube 102 to prevent axial displacement during operation. The spark screen 110 may be removable by applying an axial force of approximately 5 N to 15 N to overcome the retention mechanism, allowing access for cleaning and replacement.

The fan assembly may be configured to provide airflow characteristics that allow for use of powder form incense applications. For powder incense, the fan may operate at lower speeds to prevent dispersing unburned powder particles while maintaining adequate airflow for combustion. The airflow velocity through powder incense may be maintained in a range of approximately 0.05 m/s to 0.5 m/s to support smoldering without creating powder disturbance. The fan may include variable speed control to accommodate the different airflow requirements of powder versus stick or bundled incense forms. The airflow pattern may be designed to create gentle circulation through the powder bed rather than direct impingement that could displace the material.

The powered fan may be configured to increase smoke output per unit time by specific performance factors compared to manual techniques. The fan-assisted system may increase smoke generation rate by approximately 200% to 500% compared to natural smoldering, as measured by smoke particle density per unit volume. The increased output may be achieved by maintaining optimal oxygen supply to the smoldering material while simultaneously entraining and projecting the generated smoke. The fan may prevent the oxygen-starved conditions that typically reduce smoke output in manual smudging, where smoke tends to accumulate around the material and limit further combustion. The quantitative improvement may be measured as smoke particle count per cubic meter, with fan-assisted operation producing approximately 10\hat{ }6 to 10\hat{ }7 particles per cubic meter compared to approximately 10\hat{ }5 to 10\hat{ }6 particles per cubic meter for manual techniques under similar material and environmental conditions.

The container tube 102 may include airflow passages configured to direct air from the fan 112 through the internal chamber. The airflow passages may comprise the open cross-sectional area of the internal chamber not occupied by the incense material. The airflow passages may allow air to flow along the longitudinal axis of the container tube 102. The airflow may contact the smoldering incense material to sustain combustion and entrain smoke particles. The smoke-laden air may then flow through the spark screen 110 and exit the container tube 102.

The airflow path through the device may be configured to optimize smoke generation and projection while maintaining smoldering conditions. Ambient air may enter the housing through inlet ports positioned near the fan assembly, creating a low-pressure region that draws air through the combustion chamber. The air velocity through the combustion chamber may be maintained in a range of approximately 0.1 m/s to 2.0 m/s to provide adequate oxygen for smoldering while preventing flame re-ignition. The airflow may pass over the smoldering material in a substantially parallel direction to the longitudinal axis of the smudging material to maximize smoke entrainment. The combined air and smoke mixture may exit through the spark screen at a velocity in a range of approximately 1.0 m/s to 5.0 m/s to provide directional smoke projection. The airflow path may include smooth transitions with bend radii of at least 5 mm to minimize turbulence and pressure losses that could reduce smoke output efficiency.

The outer wall 106 of the container tube 102 may include a thermal insulation layer. The thermal insulation layer may be applied to an exterior surface of the outer wall 106. The thermal insulation layer may comprise a ceramic coating. The thermal insulation layer may comprise a polymer coating having low thermal conductivity. The thermal insulation layer may reduce heat transfer from the container tube 102 to the housing 116. The thermal insulation layer may protect a user from burns when handling the smudging device 100 during or immediately after operation.

The thermal insulation may comprise specific materials and configurations to prevent heat transfer to the user's hand during operation. The insulation may include a layer of ceramic fiber insulation having a thickness of approximately 3 mm to 10 mm positioned between the combustion chamber outer wall and the housing inner wall. The insulation material may have a thermal conductivity of less than approximately 0.1 W/m·K to limit heat transfer. The grip area may be maintained at a temperature of less than approximately 40° C. during normal operation when the combustion chamber reaches temperatures of approximately 300° C. to 400° C. The insulation may be secured using high-temperature adhesive or mechanical fasteners that do not create thermal bridges. The insulation coverage may extend from approximately 20 mm below the combustion chamber to approximately 20 mm above the chamber to provide adequate thermal protection for the entire grip region.

The container tube 102 may be configured to be easily cleaned after use. The smooth interior surfaces of the internal chamber may facilitate removal of accumulated ash and residue. The spark screen 110 may be removable to allow cleaning or replacement. The incense container 104 may be accessible for cleaning when spark screen 110 is detached.

### C. A Spark Cover

A removable cap 112 may be configured to attach to the open end of the container tube 102. The removable cap 112 may include a spark cap 114, providing secondary containment for embers and sparks. The removable cap 112 and the spark cap 114 may be constructed from heat-resistant material matching or complementing the container tube material. The spark cap 114 may include perforations or vents to allow smoke egress while preventing larger particles from escaping.

In some embodiments, the removable cap 112 may attach to the container tube via a threaded engagement mechanism. The container tube may include external threads formed on its outer surface near the open end. The removable cap 112 may include corresponding internal threads formed on its inner surface. A user may rotate the removable cap 112 relative to the container tube to engage or disengage the threaded connection. The threaded engagement may provide secure retention of the removable cap 112 during device operation. The threaded engagement may allow easy removal of the removable cap 112 for incense material loading and cleaning.

Alternatively, the removable cap 112 may attach to the container tube via a twist-lock mechanism. The container tube may include one or more tabs or projections extending radially outward near the open end. The removable cap 112 may include corresponding slots or recesses configured to receive the tabs. A user may align the tabs with the slots and insert the removable cap 112 onto the container tube. The user may then rotate the removable cap 112 through a predetermined angle to engage the tabs in locking positions within the slots. The twist-lock mechanism may provide quick attachment and detachment of the removable cap 112.

In another embodiment, the removable cap 112 may attach to the container tube via a snap-fit mechanism. The container tube may include one or more flexible detents or protrusions near the open end. The removable cap 112 may include corresponding recesses or grooves configured to receive the detents. A user may press the removable cap 112 onto the container tube with sufficient force to deflect the detents and engage them in the recesses. The snap-fit mechanism may provide audible and tactile feedback when the removable cap 112 is fully seated. The user may remove the removable cap 112 by applying sufficient pulling force to disengage the detents from the recesses.

The removable cap 112 may include a gripping surface to facilitate manual attachment and removal. The gripping surface may comprise knurling, ridges, or textured patterns formed on the outer surface of the removable cap 112. The gripping surface may enhance friction between the user's fingers and the removable cap 112. The removable cap 112 may include a flange or enlarged diameter portion to provide additional gripping area.

The spark cap 114 may include exhaust openings to direct smoke output from the device. The exhaust openings may be arranged in a predetermined pattern on the spark cap 114. As non-limiting examples, the pattern may comprise a circular array of openings around the periphery of the removable cap 112, or a central opening surrounded by smaller peripheral openings. The size and distribution of the exhaust openings may be selected to balance smoke flow rate with ember containment effectiveness.

In addition to directing smoke output, the spark cap 114 may function as a snuffing device when the exhaust openings are sealed or restricted. The spark cap 114 may include a secondary closure mechanism that can block the exhaust openings to create an oxygen-starved environment within the combustion chamber. This closure mechanism may comprise a sliding cover, rotating disk, or removable plug that can be positioned to seal the exhaust openings. When the exhaust openings are sealed, the limited oxygen supply within the sealed combustion chamber may be quickly consumed by the smoldering material, causing natural extinguishment. This snuffing capability may eliminate the need for external extinguishing methods and may provide immediate termination of the smudging process when desired.

In some embodiments, the spark cover 114 may be integrated with the removable cap 112 as a single assembly. The mesh screen or perforated barrier may be attached to the inner surface of the removable cap 112. When the removable cap 112 is attached to the container tube, the spark cover 114 may be positioned across the open end of the container tube. This integrated configuration may simplify assembly and ensure proper positioning of the spark cover 114 relative to the incense material. Alternatively, the spark cover 114 may be a separate component from the removable cap 112. The spark cover 114 may be permanently or removably installed within the removable cap. In either case, the removable cap 112 and spark cover 114 may provide secondary containment downstream of the spark screen 110. This two-stage filtration approach may enhance safety by providing redundant barriers against ember escape.

The spark cover 114 may also serve as a snuffing mechanism to extinguish the smoldering material when use is complete. When the removable cap 112 is fully attached and sealed against the container tube 102, the spark cover 114 may restrict oxygen flow to the combustion chamber, causing the smoldering material to extinguish due to oxygen depletion. The sealing element between the removable cap 112 and container tube 102 may create an airtight seal that prevents ambient air from reaching the smoldering material. This snuffing functionality may provide a safe and convenient method to terminate the smudging process without requiring water or other extinguishing agents. The user may activate the snuffing function by ensuring the removable cap 112 is fully tightened or engaged, creating complete containment of the combustion chamber.

The removable cap 112 may include a sealing element to prevent air leakage around the interface with the container tube. The sealing element may comprise an O-ring or gasket positioned in a groove on the removable cap 112 or container tube. When the removable cap 112 is attached, the sealing element may be compressed between the cap and tube surfaces to form an airtight seal. The seal may improve the efficiency of the fan-driven airflow by preventing bypass around the incense material.

### D. A Fan With Controller

As used herein, the term “fan” is intended as a general term including various air-moving devices. As examples, the term “fan” may encompass fans (e.g., axial fans, centrifugal fans, mixed flow fans, etc.), blowers, impellers, and/or any device configured to generate airflow.

The smudging device 100 may include a fan 126. The specific type of fan may be selected based on airflow requirements and/or space constraints within the housing 116. The fan 126 may be configured to generate controlled airflow through the container tube 102. As one non-limiting example, the fan 126 may comprise a motor and impeller assembly. The motor may be an electric motor powered by the power source 130. The impeller may be coupled to the motor shaft and may include blades configured to move air when rotated by the motor. The fan 126 may be positioned within the housing 116 and may be oriented to draw ambient air into the housing 116 through the airflow apertures 124 and direct the drawn air through the container tube 102.

The fan 126 may include a controller for controlling fan operation and speed. The controller may comprise electronic circuitry operatively coupled to the motor. The controller may regulate electrical power delivered to the motor. The controller may modulate motor speed by varying voltage or current supplied to the motor and/or by implementing pulse-width modulation (PWM). The controller may comprise a microcontroller or microprocessor, and a memory for storing operational parameters. The controller may execute firmware or software instructions to manage fan operation.

The fan assembly may include specific performance parameters to generate adequate airflow for smoke entrainment and projection. The fan may be configured to generate an airflow rate in a range of approximately 0.5 cubic feet per minute (CFM) to 5.0 CFM to provide sufficient air velocity for smoke transport without extinguishing the smoldering material. The fan blades may have a diameter in a range of approximately 15 mm to 40 mm and may include 3 to 7 blades with a blade angle of approximately 15 degrees to 45 degrees relative to the rotational plane. The motor may operate at a rotational speed in a range of approximately 3,000 RPM to 15,000 RPM to achieve the desired airflow characteristics. The fan assembly may be positioned at a distance of approximately 20 mm to 60 mm from the combustion chamber outlet to create adequate pressure differential for smoke entrainment. The fan housing may include inlet and outlet ducts configured to direct airflow in a substantially axial direction through the combustion chamber.

The controller may be controlled by user input via the control switch or button 128. The control switch 128 may be mounted on the housing 116 and positioned on the handle region for easy access by a user. The control switch 128 may comprise a push-button switch, toggle switch, rotary switch, slide switch, and/or other device that provides user input to the controller. The controller may receive signals from the control switch 128 and interpret the signals to determine desired fan operation.

In some embodiments, the control switch 128 may be configured as a simple on/off switch. Actuation of the control switch 128 may activate the fan 126. Deactivation of the control switch 128 may deactivate the fan 126. In other embodiments, the control switch 128 may be configured as a multi-position switch. The multi-position switch may allow selection between discrete fan speeds (e.g., low, medium, and high speed settings). The controller may adjust motor speed based on the selected switch position. In still other embodiments, the control switch 128 may be configured as a variable control, allowing continuous adjustment of fan speed. The variable control may comprise a potentiometer or capacitive touch sensor. The controller may read the variable control position and set motor speed proportional to the control position.

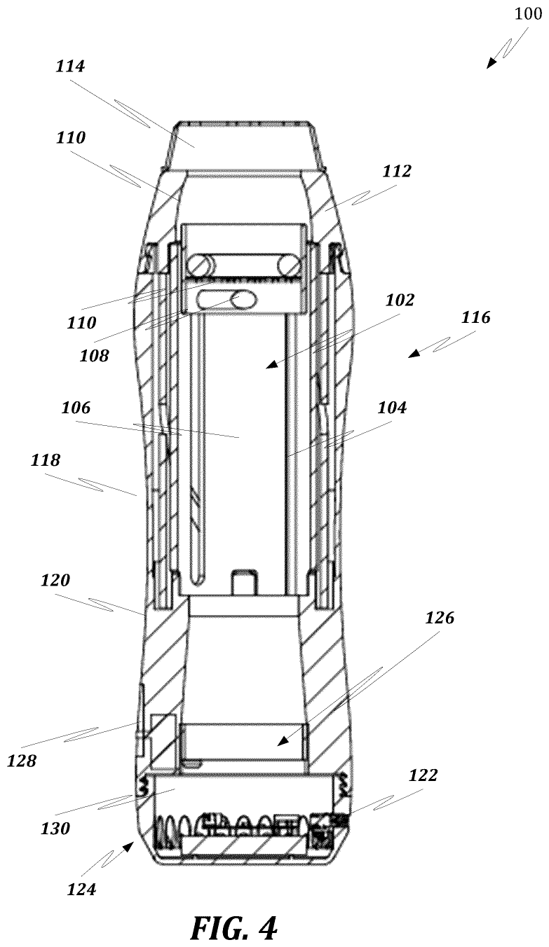

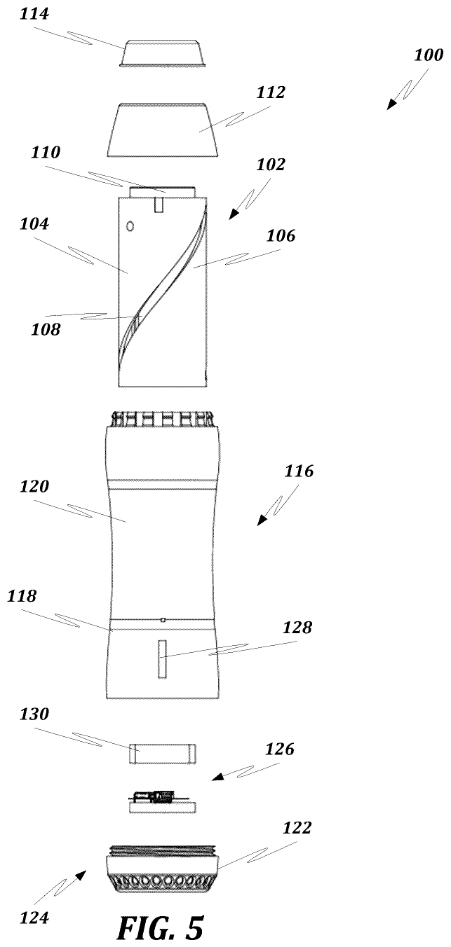

In some embodiments (as shown in FIGS. 4-5), the fan 126 and controller may be configured as an integrated unit. The integrated unit may comprise the motor, impeller, and controller housed together. The integrated unit may be a single assembly, mounted as a complete module within the housing 116. The integrated unit may simplify assembly of the device 100.

Alternatively, the fan 126 and controller may be configured as separate modules. The fan 126 may comprise the motor and impeller as a first module. The controller may be a separate second module. The first module may be mounted in a first location within the housing 116. The second module may be mounted in a second location within the housing 116. The first and second modules may be electrically connected by wiring. The separate module configuration may allow independent replacement of components and/or provide flexibility in component placement within the housing 116.

The controller may include additional features beyond basic speed control. As a first non-limiting example, the controller may implement a soft-start function, which may gradually increase motor speed upon activation, thereby reducing mechanical stress on components and reducing acoustic noise during startup. As another non-limiting example, the controller may implement a timer function, which may automatically deactivate the fan 126 after a predetermined duration. The predetermined duration may be user-selectable or factory-set. The timer function may conserve battery power and prevent unintended extended operation.

The controller may monitor operational parameters (e.g., motor current, temperature, battery voltage, etc.). The controller may detect overcurrent conditions and shut down the motor upon detecting overcurrent. The controller may monitor motor temperature (e.g., via a temperature sensor positioned near the motor) and reduce motor speed if temperature exceeds a first threshold and/or shut down the motor if temperature exceeds a critical threshold. The controller may monitor battery voltage. Responsive to detecting low battery conditions, the controller may provide a low battery indication to the user and/or reduce motor speed to extend battery life.

The mounting of the fan 126 within the housing 116 may include vibration isolation to reduce transmission of motor vibrations to the housing 116, which may help to reduce acoustic noise produced by the fan. Vibration isolation may comprise rubber grommets, foam pads, and/or resilient supports.

### E. A Power Source

The power source 130 may provide electrical energy to operate the fan 126 and other electronic components of the smudging device 100 (e.g., the controller, any LEDs, and capacitive buttons, etc.). The power source 130 may be housed within the housing 116. The power source 130 may be electrically coupled to the fan 126 through the controller. The power source 130 may be selected to provide sufficient capacity for extended operation while maintaining a compact form factor and relatively low weight suitable for handheld use.

In some embodiments, the power source 130 may comprise one or more replaceable batteries. The replaceable batteries may comprise alkaline batteries in standard battery sizes (e.g., AA, AAA, 9V, etc.). The replaceable batteries may be inserted into a battery compartment within the housing 116. The battery compartment may be accessed via a removable cover. The battery compartment may include electrical contacts that engage with terminals of the replaceable batteries to provide electrical connection between the replaceable batteries and the fan 126. The use of replaceable batteries may provide certain advantages. Replaceable batteries may allow a user to quickly restore power by installing fresh batteries. Replaceable batteries may eliminate the need for charging time, allowing the device 100 to be used immediately after battery depletion by swapping in new batteries.

Alternatively, the power source 130 may comprise one or more rechargeable batteries (e.g., lithium-ion batteries, nickel-metal hydride batteries, lithium-polymer batteries, etc.). The rechargeable batteries may be permanently installed within the housing 116. The rechargeable batteries may be removable from the housing 116 for replacement after end of service life. A rechargeable battery may provide certain advantages. A rechargeable battery may offer high energy density and a consistent voltage output throughout the discharge cycle. The battery may be recharged hundreds of times before capacity degradation becomes significant.

In embodiments where the power source 130 is a rechargeable battery, the housing 116 may include a charging port. The charging port may be located on the fixed sidewall component 118 or the base 122. The charging port may comprise a USB interface (e.g., a USB Type-C connector. a micro-USB connector, etc.), a proprietary charging connector, and/or any other connector used to transfer power to the power source 130. The charging port may be covered by a protective flap when not in use to help prevent ingress of moisture and debris into the charging port.

The charging system may comprise a dedicated charging station or cable assembly configured to interface with the handheld smudging apparatus. The charging system may include a charging base having electrical contacts positioned to align with corresponding contacts on the handheld device when placed in a predetermined orientation. The charging base may provide a charging current in a range of approximately 0.5 A to 2.0 A at a voltage compatible with the portable power source specifications. The charging system may include status indicators such as LED lights configured to display charging progress, with different colors indicating charging states such as red for charging, green for fully charged, and amber for charging faults. The charging system may include safety features such as over-current protection limiting charging current to prevent battery damage, and temperature monitoring to suspend charging if the battery temperature exceeds approximately 45° C. The charging interface may be configured for repeated connection cycles with a minimum service life of 1000 insertion and removal cycles.

The device 100 may include charging circuitry when the power source 130 comprises rechargeable batteries. The charging circuitry may be integrated into the controller, or may be a separate circuit board within the housing 116. The charging circuitry may regulate charging current and/or voltage to the rechargeable battery, and may terminate charging when the battery reaches full capacity.

The power source 130 may be selected based on expected usage patterns. For applications requiring extended continuous operation, a higher capacity battery may be selected. For applications where weight is a primary concern, a lower capacity battery with reduced weight may be selected. For applications where cost is a primary concern, replaceable alkaline batteries may be selected. For applications where convenience and environmental considerations are priorities, rechargeable batteries may be selected.

The battery power source may provide specific electrical characteristics to support fan operation for typical smudging sessions. The battery may have a capacity in a range of approximately 500 mAh to 3000 mAh to provide continuous operation for approximately 30 minutes to 4 hours depending on fan speed settings. The battery may provide a voltage in a range of approximately 3.0V to 12.0V to drive the DC motor efficiently. The battery may be a rechargeable lithium-ion type having a charge cycle life of at least 300 cycles to provide adequate service life. The battery may include integrated protection circuitry to prevent over-discharge below approximately 2.5V and over-charge above approximately 4.2V per cell. The charging interface may support charging currents in a range of approximately 0.5 A to 2.0 A to enable recharge times of approximately 1 to 6 hours. The battery may be positioned within the housing to maintain the center of gravity within the specified grip region for balanced single-handed operation.

### III. Platform Operation

The method stages disclosed herein may be performed by one or more actors, including human users, automated systems, or combinations thereof. The stages may be performed in various sequences and orders. The stages may be combined, separated, reordered, or modified without departing from the scope of the disclosed methods. Additional intermediary stages may exist between the disclosed stages. Various stages may be added or removed while still falling within the scope of the present disclosure. The methods may be implemented using hardware, software, firmware, or combinations thereof. The methods may be performed by components of the portable smoke dispersal device, by external systems in communication with the device, or by users operating the device.

The method stages may be performed in parallel or in series. Multiple stages may be performed simultaneously. The order of stages may be adjusted based on operational requirements, user preferences, or environmental conditions. The methods may be adapted for different types of incense materials, different spatial configurations, or different ceremonial practices. The methods may incorporate feedback mechanisms to adjust subsequent stages based on results of earlier stages. The methods may be automated, semi-automated, or manually controlled. The methods may be performed repeatedly in iterative cycles. The methods may be interrupted and resumed without loss of functionality.

### A. Method of Smudging a Space With Incense

Consistent with embodiments of the present disclosure, a method may be performed by at least one of the aforementioned modules. The method may be embodied as, for example, but not limited to, computer instructions, which, when executed, perform the method.

The method of smudging a space with incense may provide a comprehensive approach to space smudging that addresses the limitations of traditional manual techniques. The systematic procedure may begin with the preparation phase, where the user inserts smudging material into the combustion chamber and establishes proper smoldering conditions. The ignition and flame extinguishing steps may ensure that the material transitions to a controlled smoldering state that produces consistent smoke output without the safety hazards associated with open flames. The attachment of the spark capture screen may provide critical safety containment while maintaining smoke flow efficiency.

The operational phase of the method may involve activating the fan assembly to generate controlled airflow that entrains and directs the smoke produced by the smoldering material. This fan-assisted approach may eliminate the need for manual intervention such as repeated blowing on the incense, which can be physically demanding and may result in inconsistent smoke production. The directional smoke output capability may enable users to target specific areas within a space, including corners, ceiling perimeters, and areas behind furniture that may be difficult to reach with naturally rising smoke from traditional methods. The method may provide enhanced coverage efficiency and may reduce the time required to complete smudging ceremonies in larger spaces.

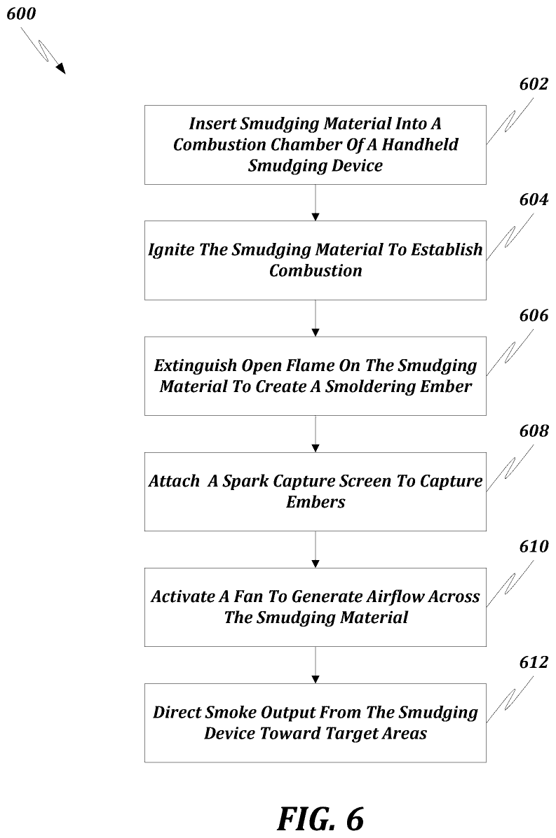

FIG. 6 is a flow chart setting forth the general stages involved in a method 600 consistent with an embodiment of the disclosure for a method of smudging a space with incense using the smudging device 100. The method 600 may comprise a series of stages performed using the portable smoke dispersal device 100. Each stage may be performed sequentially to achieve controlled smoke generation and distribution throughout a target space. The method 600 may be adapted for various types of incense materials and different spatial configurations.

The method 600 may begin at stage 602, where a user may insert smudging material into the combustion chamber of the handheld smudging device 100. The smudging material may comprise sage, palo santo, cedar, sweetgrass, or other ceremonial incense materials. The smudging material may be provided in stick form, in bundle (e.g., “smudge stick”) form, in a loose form, in powder form, and/or in other forms that can be disbursed via smoldering.

To insert the smudging material, the user may access the combustion chamber. In some embodiments, accessing the combustion chamber may be accomplished by removing the removable cap 112 from the container tube 102. The removal may be accomplished by rotating the removable cap 112 in a first direction if a threaded engagement is used, pulling the removable cap away from the container tube 102 if a friction fit is used, or rotating and pulling the removable cap 112 if a twist-lock mechanism is used. The user may further remove the spark screen 110 to expose the combustion chamber of the incense container 104. In other embodiments, the user may be able to access the combustion chamber without removing the cap 112.

The step of inserting smudging material into the combustion chamber may involve specific procedures to ensure proper positioning and secure retention. The smudging material may be inserted by first removing the spark capture screen to provide access to the combustion chamber opening. The material may be inserted to a depth of approximately 10 mm to 50 mm into the chamber, depending on the material length and desired burn duration. For bundled materials such as sage, the bundle may be compressed slightly during insertion to ensure contact with the chamber retention features. The insertion force may be approximately 2 N to 8 N to overcome the friction-fit retention mechanism without damaging the material. The material may be oriented with the intended burning end positioned toward the chamber outlet to optimize airflow interaction and smoke generation.

For powder form incense applications, the insertion step may involve pouring or spooning the powder material into the combustion chamber through the access opening. The powder may be distributed evenly across the bottom of the container to ensure uniform burning and consistent smoke production. The powder depth may be controlled to maintain proper airflow while providing adequate material for the desired burn duration. The powder form may include traditional materials such as ground sage, powdered palo santo, cedar dust, or commercial incense powders specifically formulated for smudging applications. The container design may accommodate powder forms ranging from fine dust particles to coarse granules.

The user may insert the smudging material through the open end of the incense container 104. The smudging material may be inserted until a first end of the smudging material contacts an internal support structure within the incense container 104. The internal support structure may comprise friction-fit surfaces. The internal support structure may comprise mechanical retention features. The smudging material may be positioned such that a second end of the smudging material extends toward the open end of the container tube 102.

The positioning of the smudging material may optionally be adjusted by rotating the rotatable adjustment component 112 of the housing 116. The rotation may cause axial translation of the container tube 102 relative to the housing 116 when adjustment is desired. The optional axial translation may move the smudging material closer to or farther from the spark cover 110. The adjustment may provide optional optimization of the position of the smudging material relative to the airflow path generated by the fan 126. The device may function effectively with the container tube 102 in a fixed position, with the adjustment capability serving as an optional enhancement for specific applications or user preferences.

The rotatable adjustment component 112 may provide optional positioning control rather than required adjustment for device operation. The device 100 may function effectively with the container tube 102 maintained in a default or fixed position without requiring translational movement. The optional adjustment capability may be provided for users who desire to fine-tune the material position for specific smudging materials, burn characteristics, or personal preferences. The device may include detents or position indicators to establish preferred positions for the container tube 102, allowing users to return to optimal settings or to operate without adjustment if desired.

The method 600 may proceed to stage 604, where the smudging material may be ignited to establish combustion. The ignition may be performed using a flame source, such as a lighter, a match, a torch, any other device capable of producing an open flame, an electric plasma arc lighter, and/or the like.

The step of igniting the smudging material may be performed using conventional ignition sources while following specific safety procedures. The material may be ignited using a lighter, match, or other flame source applied to the exposed end of the material for approximately 10 to 30 seconds until sustained combustion is established. The ignition may be confirmed by observing a steady flame of approximately 5 mm to 20 mm in height extending from the material surface. During ignition, the spark capture screen may remain removed to provide access and prevent heat buildup. The ignition process may be performed in a well-ventilated area away from flammable materials, with the device held at arm's length to prevent exposure to ignition flames.

The burning process may alternatively be started by application of a heated electrical coil to the incense container 104 and/or the material within the container. The electrical coil may be integrated as part of the device 100 or may be provided as a separate ignition device. When integrated into the device 100, the electrical coil may be positioned within or adjacent to the combustion chamber and may be powered by the same battery power source that operates the fan assembly. The coil may be configured to heat to a temperature in a range of approximately 300° C. to 600° C. to initiate combustion of the smudging material. For powder material applications, the heated coil may be positioned to contact the powder directly or may heat the container walls to initiate smoldering through conductive heat transfer.

The electrical coil ignition method may involve activating the heating element and allowing it to reach operating temperature before applying it to the material or container. For integrated coil systems, the user may activate the heating function using the device controls and wait for the material to begin smoldering before activating the fan assembly. For separate coil devices, the heated coil may be applied to the material for approximately 10 to 60 seconds until smoldering is established, after which the coil device may be removed and the fan activated. The electrical coil method may be particularly effective for powder materials that may be difficult to ignite with open flame, providing controlled and consistent ignition without the safety concerns associated with flame sources.

The user may apply the ignition source to the second end of the smudging material. The second end may be the end extending toward the open end of the container tube 102. The ignition source may be held in contact with the smudging material for a duration sufficient to establish combustion (e.g., in a range of approximately 5 seconds to 30 seconds). For example, the duration may be in a range of approximately 10 seconds to 15 seconds for sage bundles, or approximately 15 seconds to 20 seconds for palo santo sticks.

The combustion may be characterized by a visible flame on the surface of the smudging material. The flame may propagate across the exposed surface of the smudging material, generating heat that initiates thermal decomposition of the plant material. The thermal decomposition may release volatile organic compounds, including aromatic oils characteristic of the smudging material.

The user may observe the flame to ensure adequate combustion has been established, including verifying that the flame has spread across a sufficient area of the smudging material surface and is self-sustaining without continued application of the flame source. The user may remove the flame source once adequate combustion has been established.

In stage 606, the method 600 may comprise extinguishing the open flame from the smudging material while maintaining smoldering. The extinguishment may be performed after the predetermined time period has elapsed. The extinguishment may transition the combustion from flaming combustion to smoldering combustion, characterized by flameless oxidation of the smudging material.

The user may extinguish the flame by blowing on the burning smudging material. The blowing may direct a stream of air across the flame that cools the combustion zone below the temperature required to sustain flaming combustion. Alternatively, the user may extinguish the flame by waving the device 100 through the air. The waving motion may create relative air movement across the flame, similar to the blowing action. In still other embodiments, the user may extinguish the flame by briefly covering the open end of the container tube 102, restricting oxygen supply to the combustion zone.

The step of extinguishing open flame while maintaining smoldering may be accomplished through controlled airflow restriction or gentle agitation. The open flame may be extinguished by gently blowing on the burning material or by briefly restricting airflow to reduce oxygen supply. The extinction process may take approximately 5 to 15 seconds and may be confirmed by the absence of visible flame while maintaining a glowing ember at the material surface. The smoldering state may be characterized by the presence of visible smoke generation and a surface temperature of approximately 300° C. to 500° C. at the burning end. The transition from flame to smoldering may be critical to prevent re-ignition during subsequent fan operation while maintaining adequate heat for sustained smoke production.

After extinguishment of the flame, the smudging material may continue to smolder. The smoldering may be characterized by a glowing ember at the surface of the smudging material. The ember may have a temperature in a range of approximately 500° C. to 700° C. The smoldering may generate smoke without producing a visible flame. The smoke may comprise fine particles (the aromatic compounds volatilized from the smudging material) suspended in air.

The user may verify that smoldering has been established by observing visible smoke rising from the smudging material and a glowing ember on the surface of the smudging material. The verification may comprise detecting the characteristic scent of the smudging material. If smoldering has not been established, the user may return to the ignition stage 604.

If smoldering is established in stage 606, the method 600 may proceed to stage 608, where one or more spark capture screens may be installed over the combustion chamber. The spark capture screen may be the spark screen 110 and/or the removable cap 112 including the spark cap 114. The spark screen 110 and/or spark cap 114 may comprise perforated barriers configured to capture embers and/or ash while allowing smoke passage.

The step of attaching the spark capture screen may involve specific alignment and securing procedures to ensure proper containment. The screen may be positioned over the combustion chamber outlet by aligning any orientation features such as tabs or notches with corresponding features on the chamber. The screen may be pressed into position with a force of approximately 5 N to 12 N until the retention mechanism engages, which may be confirmed by a tactile click or resistance increase. The proper attachment may be verified by ensuring the screen is flush with the chamber rim and cannot be displaced by gentle lateral force. The attachment process may be performed while the material is in the smoldering state, requiring care to avoid contact with hot surfaces.

To install the spark screen 110, the user may place the spark screen within the container tube 102. The spark screen 110 may divide the tube into the combustion chamber containing the smudging material and a smoking transmission chamber above the spark screen. The spark screen may be held in place by a friction fit, or may snap into place within an annular groove in the container tube.

To install the spark cap 114, the user may align the removable cap 112 with the open end of the container tube 102. The alignment may involve positioning tabs on the removable cap 112 with corresponding slots on the container tube 102 if a twist-lock mechanism is used. The alignment may involve positioning threads on the removable cap 112 with corresponding threads on the container tube 102 if a threaded engagement is used.

The user may attach the removable cap 112 to the container tube 102. The attachment may be accomplished by rotating the removable cap 112 in a second direction opposite the first direction if a threaded engagement is used. The rotation may continue until the removable cap 112 is fully seated against the container tube 102. The attachment may be accomplished by pressing the removable cap 112 onto the container tube 102 if a snap-fit mechanism is used. The pressing may continue until an audible click indicates full engagement.

The attachment may create a seal between the removable cap 112 and the container tube 102. The seal may prevent embers from escaping around the interface. The seal may be formed by a sealing element. The sealing element may comprise an O-ring or gasket that is compressed when the removable cap 112 is fully attached.

As a part of the removable cap 112, 5he spark cap 114 is positioned across the open end of the container tube 102 when the removable cap is attached. The spark cap 114 may provide secondary containment for embers, while exhaust openings allow smoke to exit and prevent ember passage.

This dual-layer of spark prohibition may help to prevent accidental disbursement of sparks or ash during the smudging process.

The method 600 may proceed to stage 610, which may include activating a fan 126 within the handheld smudging device 100 to generate airflow across the smoldering smudging material. The activation may be performed by actuating the control switch 128 mounted on the housing 116.

The user may press the control switch 128 to activate the fan 126. The pressing may close an electrical circuit between the power source 130 and the fan 126. The closed circuit may allow electrical current to flow from the power source 130 to the motor of the fan 126. The electrical current may energize the motor. The energized motor may rotate the impeller of the fan 126.

The rotating impeller may draw ambient air into the housing 116 through the airflow apertures 124. The ambient air may enter the housing 116 at the base 122 and flow upward through the housing 116 toward the fan 126. The fan 126 may accelerate the ambient air and direct the air through the container tube 102.

The airflow may pass through the container tube 102 along the longitudinal axis, and may contact the smoldering smudging material. The contact may sustain the smoldering combustion by providing oxygen, thus maintaining smoke generation.

The airflow may entrain smoke particles generated by the smoldering smudging material. The entrainment may occur as the moving air carries the smoke particles along with it. The entrained smoke may flow through the spark screen 110, while the spark screen may capture embers and/or ash entrained in the airflow. The smoke may then flow through the spark cap 114. The spark cap 114 may provide additional ember and/or ash filtration.

The smoke may exit the device 100 through the exhaust openings in the spark cap 114. The exiting smoke may form a directed stream having a velocity higher than smoke produced by natural smoldering without fan assistance. The higher velocity may enable the smoke to travel farther from the device 100.

The user may adjust the fan speed by manipulating the control switch 128. The controller operatively coupled to the fan 126 may regulate the fan speed based on the position of the control switch 128. The controller may adjust the voltage and/or current supplied to the fan motor, or implement pulse-width modulation to control the fan speed. The pulse-width modulation may vary the duty cycle of power delivery to the motor.

The method 600 may continue to stage 612, where the user may direct smoke output from the handheld smudging device 100 toward target areas within the space. The directing may be accomplished by manually aiming the device 100. The user may grasp a handle portion of the housing 116, and may point the open end of the container tube 102 toward a first target area. The first target area may be a corner of the space, a doorway, a window, and/or the like. The directed smoke stream may travel from the device 100 to the first target area.

The step of directing smoke output may involve specific positioning and movement techniques to achieve effective space coverage. The device may be held at approximately arm's length from the user and oriented to direct smoke toward target areas at angles ranging from horizontal to approximately 45 degrees upward to account for natural smoke rise. The device may be moved in slow, sweeping motions at a rate of approximately 10 cm/s to 50 cm/s to distribute smoke evenly across target surfaces. For corner treatment, the device may be positioned approximately 30 cm to 100 cm from the corner and angled to direct smoke into the corner junction. The smoke projection distance may be approximately 0.5 m to 3.0 m depending on fan speed and ambient air conditions, allowing coverage of typical room dimensions without requiring user movement throughout the entire space.

The user may maintain the device 100 aimed at the first target area for a first duration. The first duration may be sufficient to achieve desired smoke coverage of the first target area (e.g., approximately 5 seconds to 30 seconds). The first duration may vary based on the size of the first target area and/or the desired smoke density.

The user may move the device 100 to aim at a second target area. The movement may be a sweeping motion to transition the smoke stream from the first target area to the second target area. The user may repeat the aiming and dwelling process for the second target area.

The user may systematically traverse the space while operating the device 100. The traversal may follow a predetermined pattern (e.g., a clockwise path around the perimeter of each room. a path that visits each corner of each room, a path that covers high areas before low areas, etc.). The systematic traversal may ensure comprehensive coverage of the space.

The user may adjust the fan speed during the directing stage. The adjustment may be based on the characteristics of the current target area. The user may increase the fan speed when directing smoke toward high ceiling areas. The increased fan speed may provide greater smoke velocity to reach the high areas. The user may decrease the fan speed when directing smoke toward smaller or more enclosed areas. The decreased fan speed may prevent excessive smoke accumulation.

The user may monitor the smoke output during the directing stage. The monitoring may include observing the visibility of the smoke stream. The monitoring may include observing the distance the smoke travels from the device 100. The monitoring may include detecting the scent intensity in different areas of the space. If the smoke output diminishes, the user may verify that the smudging material is still smoldering. If the smudging material has extinguished, the user may return to the ignition stage.

The directing stage may continue until the desired coverage of the space has been achieved. The desired coverage may be subjective based on the user's ceremonial and/or practical objectives. The desired coverage may be objective based on predetermined criteria such as smoke visibility in all areas. The directing stage may continue until the smudging material is substantially consumed. The consumption may be indicated by reduced smoke output despite continued fan operation.

Upon completion of the directing stage, the user may deactivate the fan 126. The deactivation may be accomplished by actuating the control switch 128 to an off position. The deactivation may stop the airflow through the container tube 102. The smoldering smudging material may continue to generate smoke after fan deactivation. The smoke generation may gradually diminish as the ember cools.

The user may allow the smudging material to extinguish naturally. The natural extinguishment may occur as the ember consumes the remaining combustible material, and/or as the ember cools below the temperature required to sustain smoldering. Alternatively, the user may actively extinguish the smudging material. The active extinguishment may be accomplished by removing the removable cap 112 and smothering the ember (e.g., using water, sand, salt, and/or or another non-combustible material).

The spark cover system may provide an integrated snuffing mechanism that allows immediate extinguishment of the smoldering material. By ensuring the removable cap 112 is fully sealed against the container tube 102, the user may create an oxygen-depleted environment that naturally extinguishes the smoldering material within approximately 30 seconds to 2 minutes. This snuffing method may be safer than water-based extinguishing, which can create steam and thermal shock, and may be more convenient than waiting for natural extinguishment. The snuffing functionality may be particularly useful in situations where immediate termination of smoke production is required, such as when moving between spaces or when the ceremony is interrupted.

After the smudging material has been extinguished and the device 100 has cooled, the user may perform maintenance. The maintenance may include removing the removable cap 112. The maintenance may include removing spent smudging material from the incense container 104, cleaning the spark screen 110 to remove accumulated ash, and/or cleaning the spark cap 114 to remove accumulated residue. The maintenance may prepare the device 100 for subsequent use.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

1. A handheld smudging device comprising:
a housing configured for single-handed operation;
a combustion chamber disposed within the housing and configured to receive smudging material;
a spark capture screen positioned to cover an outlet of the combustion chamber, the spark capture screen configured to retain burning particles while allowing smoke to pass therethrough;
a fan assembly disposed within the housing and configured to generate airflow through the combustion chamber and past the smudging material; and
a battery power source configured to power the fan assembly.

2. The handheld smudging device of claim 1, wherein the combustion chamber comprises retention features configured to secure the smudging material in a predetermined position within the combustion chamber.

3. The handheld smudging device of claim 1, wherein the spark capture screen is removably attachable to the combustion chamber to allow access for loading and unloading the smudging material.

4. The handheld smudging device of claim 1, wherein the fan assembly comprises a DC motor and fan blades configured to draw ambient air into the housing and direct the air across the smudging material.

5. The handheld smudging device of claim 1, wherein the housing comprises thermal insulation positioned between the combustion chamber and a grip area to prevent heat transfer to a user's hand.

6. The handheld smudging device of claim 1, further comprising a user interface disposed on the housing and configured to control operation of the fan assembly.

7. The handheld smudging device of claim 6, wherein the user interface comprises a multi-speed selector configured to adjust airflow velocity generated by the fan assembly.

8. The handheld smudging device of claim 1, wherein the combustion chamber is configured to maintain smoldering of the smudging material without re-igniting open flame during fan operation.

9. The handheld smudging device of claim 1, wherein the spark capture screen comprises a mesh having apertures sized to prevent passage of burning fragments while maintaining acceptable pressure drop for smoke flow.

10. The handheld smudging device of claim 1, wherein the battery power source comprises a rechargeable battery and the housing includes a charging interface.

11. A smudging system comprising:
a handheld smudging apparatus comprising:
a housing configured for portable operation;
a tubular combustion container configured to receive smudging material;
a removable spark containment cover configured to attach to the tubular combustion container;
a powered fan configured to generate directional airflow through the tubular combustion container; and
a portable power source; and
a charging system configured to recharge the portable power source.

12. The smudging system of claim 11, wherein the tubular combustion container comprises heat-resistant material and includes air inlet and outlet paths for controlled airflow.

13. The smudging system of claim 11, wherein the removable spark containment cover comprises a mesh screen configured to trap sparks and embers while allowing smoke passage.

14. The smudging system of claim 11, further comprising interchangeable combustion containers configured for different smudging materials.

15. The smudging system of claim 11, wherein the powered fan is configured to increase smoke output per unit time compared to manual smudging techniques.

16. A method of smudging a space comprising:
inserting smudging material into a combustion chamber of a handheld smudging device;
igniting the smudging material to establish combustion;
extinguishing open flame from the smudging material while maintaining smoldering;
attaching a spark capture screen over the combustion chamber;
activating a fan within the handheld smudging device to generate airflow across the smoldering smudging material; and
directing smoke output from the handheld smudging device toward target areas within the space.

17. The method of claim 16, wherein directing smoke output comprises orienting the handheld smudging device to project smoke toward corners, ceiling perimeters, and doorways.

18. The method of claim 16, further comprising adjusting fan speed to control smoke volume and projection distance.

19. The method of claim 16, wherein the smudging material comprises at least one of sage, palo santo, cedar, and sweetgrass.

20. The method of claim 16, further comprising monitoring smoke output and re-igniting the smudging material when smoke production diminishes while maintaining the spark capture screen in position during re-ignition.
